(19) United States
(12) Patent Application Publication
(10) Pub. No.: US 2021/0282976 A1
(43) Pub. Date: Sep. 16, 2021

(54) Dome Bandage

(71) Applicant: Hakeem Alraheem, Turlock, CA (US)

(72) Inventor: Hakeem Alraheem, Turlock, CA (US)

(21) Appl. No.: 17/179,169

(22) Filed: Feb. 18, 2021

Related U.S. Application Data
(60) Provisional application No. 62/990,289, filed on Mar. 16, 2020.

(51) Int. Cl. A61F 13/00

(52) U.S. Cl. CPC A61F 13/00021; A61F 2013/00272; A61F 2013/00165; A61F 2013/00182

(57) ABSTRACT

A dome bandage is a reusable device for covering wounds, optical viewing, and protecting from external objects while wound heals. In one form, the dome bandage is composed of clear transparent synthetic ultraviolet resistant rigid shatter proof reusable shell dome with size and shape in degrees from circular to ellipticity; and from squareness to oblongness mold; with slits on the flange for attaching porous non-adhesive ribbon tape to rap around the uninjured part of the human anatomy.

### CROSS-REFERNCE TO RELATED APPLICATIONS

Current US Class: 602/41 602/42; 602/60; 359/361; D24/100

Current CPC Class: A61F 13/00017;
A61F 2013/00157;
A61F 2013/00276;
A61F 2013/00404;
A61F 2013/00165;
A61F 2013/00455;
A61F 2013/00387;
A61F 2013/00204;
A61F 2013/00395;
A61F 13/49004;
A61F 2013/15276;
A61F 13/15268;
A61F 13/0286;
A61F 2013/00846;
A61F 2013/00825;
A61F 2013/00506;
A61F 2013/0051;
A61F 2013/00102;
A61F 2013/00093;
A61F 13/00025;
A61F 2013/00723;
A61F 2013/00889;
A61F 2013/00412;

U.S. Patent Documents: U.S. Pat. No. 4,285,338A 1981 Aug. 25 Lemelson Jerome H
U.S. Pat. No. 2,443,140A 1948 Jun. 8 Robert E Larsen
U.S. Pat. No. 3,416,525A 1968 Dec. 17 George J Yeremian
U.S. Pat. No. 4,023,569A 1977 May 17 Herbert WarneckeAdolf Lindner
U.S. Pat. No. 2,632,443A 1953 Mar. 24 Eleanor P Lesher
U.S. Pat. No. 3,334,626A 1967 Aug. 8 Schimmel Madalyn Miller
U.S. Pat. No. 1,616,156A 1927 Feb. 1 Vroom Edward
U.S. Pat. No. 1,956,695A 1934 May 1 Reinitz Emil
U.S. Pat. No. 3,874,387A 1975 Apr. 1 Pasquale P Barbieri
U.S. Pat. No. 2,785,677A 1957 Mar. 19 Lawrence R Stumpf
U.S. Pat. No. 4,667,666A 1987 May 26 Alice Fryslie

### BACKGROUND OF THE INVENTION—THE DOME BANDAGE

Removing bandage padding is very painful if conventional skin wound bandage padding adhesive materials are applied on top of an injured skin. The bandage padding absorbs excess blood and platelets in the wound and eventually fuses and hardens the scab to the padding. When lifting the bandage padding after the blood had dried, and hardened on the affected wound, the injured skin will rupture; disrupt the scab causing pain and will require the scab wound to re-harden for repeated proper healing. The adhesive bandage materials used to append the bandage around the wound, creates an air locked seal between the skin and the chemical bonding materials clogs skin pores. When lifting the appended adhesive bandage materials from skin tissue; attached hair follicles will be pulled from skin causing pain when the said adhesive is removed.

Conventional skin wound bandage padding material applied on top of an injured skin, does not offer impact protection for wounds if padding creases or crushes on impact. If physical contact is made by an external object on conventional skin wound bandage padding, the said bandage padding materials will crease or crush an enclosed skin wound, causing pain and scab damage.

Inspecting and viewing skin wounds from a conventional skin wound bandage, requires the removal of the bandage padding material applied on top of the injured skin. As previously stated, removing the padding for inspecting and viewing the skin wound, will be very painful to the injured victim.

Conventional skin wound bandage padding material applied on top of the injured skin, does not offer visual indication as to when the said bandage should be temporarily removed for inspections, and or permanently removed for proper healing.

Conventional skin wound bandage padding material applied on top of the injured skin, was never intended be viewed, unless the padding from the wound, was removed for wound viewing. As previously stated, removing the padding for inspecting and viewing the skin wound, will be very painful to the injured victim.

Conventional skin wound bandage padding material applied on top of the injured skin, blocks the wound from having direct and indirect exposures to natural light and air; and skin adhesive bonding materials clogs skin pores, and will also block and prevent uninjured natural skin from natural skin gas exchanges.

Conventional skin wound bandage padding material applied on top of the injured and uninjured skin, are indicative to blocking skin from having exposures to air, and sunlight. Such skin blocking wound padding material, also prevents natural skin from natural skin gas exchanges, and the said padding will cause the skin to become pale from lack of air and sunlight. For proper skin health, the skin must have proper air and sunlight exposure.

Conventional skin wound bandages are not reusable, and skin wound bandages are improperly discarded, padding with blood or wound secretion materials, can become bio-hazard risk.

Through the succeeding years, variants were presented in an attempt to alleviate these problems listed. There were many variations, including the use of dome or cup-shaped covers secured by separate adhesive means as shown in Lesher, (U.S. Pat. No. 2,632,443), Schimmel, (U.S. Pat. No. 3,334,626) and Connally, (U.S. Pat. No. 1,616,156). Other variations such as that described in Reinitz, (U.S. Pat. No. 1,956,695) involved transparent impermeable device secured by an adhesive strip for collecting secretions while Barbieri (U.S. Pat. No. 3,874,387) disclosed a similar device which included fluid valves. Stumpf, (U.S. Pat. No. 2,785,677) and Schaar, (U.S. Pat. No. 4,212,296) each disclose a protective device having a domed portion but each suffered from a allowing wound natural flowing air effect whereby bacteria will grow instead of healing, and the wound will become infected. Fryslie, (U.S. Pat No. 4,667,666) is a disposable device with foraminous vents on top of protective layer used to cover the wound, however such options in using foramious vents will weakens the structure and makes it easy to crack or break on external impact. Foraminous vents, allow liquids, dust and debris to pass through the top of the said device and thus, will not aid in blocking contaminates from entering directly making contact with wound, from top of the said device vents. Also the said device flange uses a bonding adhesive compound to stick to the skin, thus removing the device; hair follicles will be pulled from the roots causing pain, and since the device was not designed for real-time wound viewing or observation without removing the said device, the user must remove the device to inspect the wound. Lemelson, (U.S. Pat. No. 4,285,338) discloses touching the wound with absorbent materials and enclosed with an opaque hard cover and held by adhesive tape. Furthermore, none of the prior devices were able hold on the skin without an adhesive compounds and such compounds blocks the skin and wound from having proper natural skin gas exchanges, airflow protection, and sunlight.

As previously stated, adhesive compounds on skin, clogs skin pores and prevents healthy or injured skin from natural skin gas exchanges, and the said adhesive compound leaves a residue on the skin, and film substances sticks to hair follicles, collects debris, oils and unsanitary partials from external sources. Furthermore, not one of the mentioned devices collectively or individually resolve problems mentioned with regards to adhesive bonding materials. Neither of the mentioned devices addresses adjustability of their devices in establishing optimal comfort while on extremities, and adhesiveness weakens or will eventually fail to adhere to the skin if removed and readjusted on the skin. Neither of the mentioned devices addressed photosensitive skin wounds protection nor supporting ultraviolet resistant materials. Neither devices are reusable and must be discarded after use. Lastly, neither the mentioned devices addressed how long the device are to remain on the skin or the conditions of which to replace, or permanently remove the said device from use.

The present invention solution is a reusable synthetic hard ultraviolet resistant clear transparent dome cover, placed over the skin wound without any part of the said dome device touching the skin wound, and without the risk of pain caused by the said dome device; and the said device is held on the skin by porous natural skin gas exchanges non-adhesive ribbon tape to be fitted and adjustably positioned on to the uninjured part of the human anatomy, and the said device remains on the body until the wound heals, without tape adhesives or bonding materials to clogs skin pores.

Conventional skin wound bandage and current patent devices padding material are applied on top of the injured skin will touch and adhere to the wound. The benefits in comparison with the skin wound enclosed by the said ultraviolet resistant synthetic hard clear transparent dome cover device, the said device hollow area will not allow contact to the wound and is indicative in circumventing the problems associated to materials touching the wound.

Conventional skin wound bandage and current patent devices padding material are applied on top of the injured skin prevents the wound from being viewable. The benefit in comparison with the skin wound enclosed by the said ultraviolet resistant synthetic hard clear transparent dome cover device, the skin wound is always ocularly viewable and clearly visible without needing to remove the said device until the wound is completely healed.

Conventional skin wound bandage and current patent devices padding material are applied on top of the injured skin wound covered, and natural exchanges of skin gases are blocked. The benefit in comparison with the skin wound enclosed by the said ultraviolet resistant synthetic hard clear transparent dome cover device is not hermetic, and yet the device maintains moister while natural skin gas exchanges inside the device, and from the flange.

Conventional skin wound bandages and current patent devices requires disposal of used bandages and may be hazardous if materials not properly treated or disposed. The benefit in comparison with the skin wound enclosed by the said ultraviolet resistant synthetic hard clear transparent dome cover device does not touch the wound, and the said device is durable for repeated reuse after aseptic, and or sterilization methods.

Conventional skin wound devices and current patent devices require skin pore clogging materials to hold on to the skin. The benefits in comparison with the skin wound enclosed by the said ultraviolet resistant synthetic hard clear transparent dome cover device does not support bonding adhesives nor requires adhesive tape. The slits on the flange supports the porous natural skin gas exchanges non-adhesive ribbon tape to securely fasten to the said device, and securely fasten to the uninjured part of the human anatomy.

Conventional skin wound devices and current patent devices require adhesive bonds or adhesive tape to be bonded to the skin. Once the said adhesive bonds are affixed on the skin, the device is not adjustable, and upon any relocation of the said device, the adhesive materials bond weakens. The benefits in comparison with the skin wound enclosed by the said ultraviolet resistant synthetic hard clear transparent dome cover device, the non-adhesive tape straps are adjustable to suit the comfort of the injured person and can be adjusted any number of times without losing its effectiveness to append on the skin.

Conventional skin wound devices and current patent devices require adhesive bonds or adhesive tape to be bonded to the skin. Once the said adhesive bonds are affixed on the skin, the sticky film attaches to debris, skin oils, where bacteria and fungus spores can accumulate and grow. The benefits in comparison with the skin wound enclosed by the said ultraviolet resistant synthetic hard clear transparent dome cover device, non-adhesive tape straps are not held on the skin by sticky film adhesives, therefore it is easy to wipe clean any oils or unsanitary debris from non-adhesive tape.

Conventional skin wound devices and current patent devices does not offer ultraviolet resistant materials nor protect photosensitive skin wounds from sunlight ultraviolet rays. The benefits in comparison with the skin wound enclosed by the said ultraviolet resistant synthetic hard clear transparent dome cover device is ultraviolet resistant and protects photosensitive skin wounds from sunlight ultraviolet rays.

### BRIEF DESCRIPTION OF THE SEVERAL VIEWS OF THE DRAWING(S) OF THE DOME BANAGE

In the drawings:

FIG. 1 Cross sectional view.

1 Exterior wall.

1a Interior wall.

1b Ultraviolet resistant clear transparent synthetic material.

2 Flange.

FIG. 2 Bottom view.

1 Exterior wall.

1a Interior wall.

2 Flange.

3 Slit.

FIG. 3 Frontal view.

1 Exterior wall.

2 Flange.

3 Slit.

FIG. 4 Frontal view.

1 Exterior wall.

2 Flange.

3 Slit.

FIG. 5 Top view.

1 Exterior wall.

2 Flange.

3 Slit.

4 Porous non-adhesive ribbon tape.

### DETAILED DESCRIPTION OF THE DOME BANDAGE.

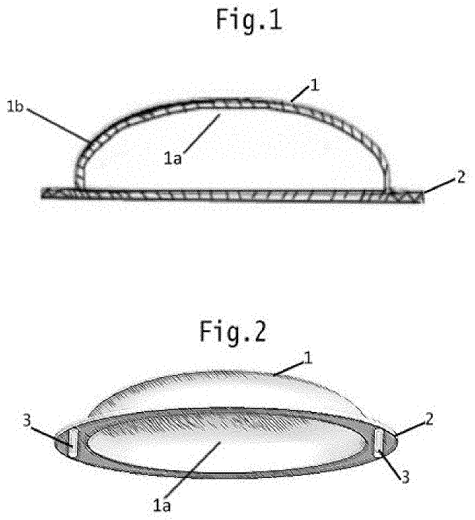

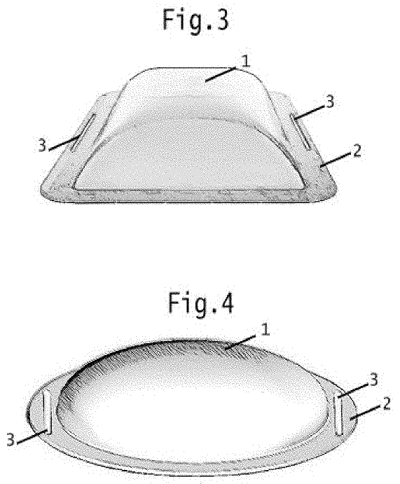

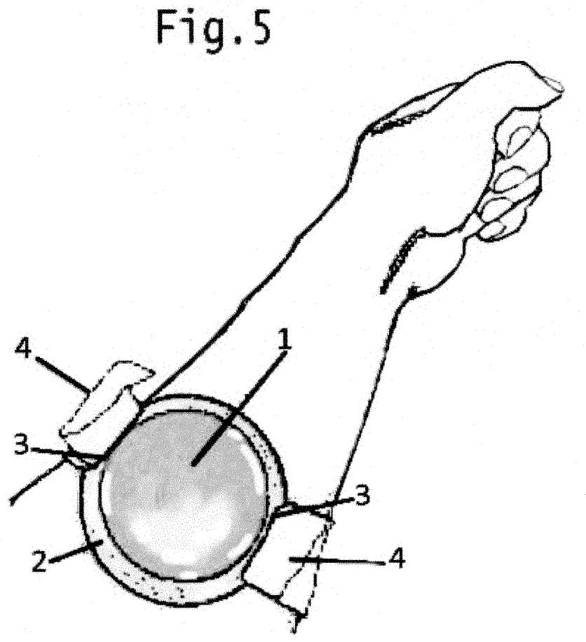

The present invention relates to a reusable device indicated at FIG. 1, FIG. 2, FIG. 3, FIG. 4 and FIG. 5 for burns, rashes, cuts, impact injuries, abrasions, punctures, ulcers, chafed skin, sutures, tattoos, warts, blisters and insect lesions wounds. While the device is in use, it does not to touch the wound; and the device also acts as a physical barrier and shield to prevent external objects from making contact to the affected area. The entire device is translucent clear ultraviolet resistant synthetic material FIG. 1-1b. The exterior wall allows clear and transparent ocular, optical viewing FIG. 1-1a which allows real time wound inspection and examination. Viewing a wound while covered and shielded allows viewing without removing the device while the wound heals completely, and without being disturbed. The said device is ultraviolet resistant synthetic materials FIG. 1-1b for withstanding sun degradation. The said device exterior wall, blocks, shields against external ultraviolet light FIG. 1-1, and -1b.

The slits indicated at FIG. 2-3; FIG. 3-3; FIG. 4-3 and FIG. 5-3; The porous natural skin gas exchanges non-adhesive ribbon tape indicated at FIG. 5-4; where the said ribbon tape is threaded through the slit, and securely applied around extremity and threading though the next adjoining slit while holding the device on top the epidermis while the said device covers, and protect the wound. While the said device is in usage, the adjustable non-adhesive ribbon tape will allow natural skin gas exchanges.

The flange bottom at FIG. 1-2; FIG. 2-2; FIG. 3-2; FIG. 4-2 and FIG. 5-2 is the only part of the device that rest on the non-injured epidermis. As shown in FIG. 1-1, 1a and 1b the ultraviolet clear transparent synthetic material will allow wound observation, and are clear from any materials that obstruct viewing. The flange FIG. 1-2; FIG. 2-2; FIG. 3-2; FIG. 4-2 and FIG. 5-2 favorably will not contour to the curvature of the extremities and; the said flange FIG. 2-2; FIG. 3-2; FIG. 4-2 and FIG. 5-2 will not create a hermetic seal which supports air ventilation and while the said device is in use, external atmosphere is indicative to equaling the interior. Furthermore, the hard-shell integrity and strength is shatter proof, thus protecting the wound during a collision with external object. Natural venation and air flow is allowed from under the flange, where air and natural skin gas exchanges will naturally circulate and pass through under the flange, and outside the chamber while the device is appended on the uninjured epidermis.

The said device geometric shape in FIG. 3 shows the degrees of square to oblongness supporting wounds of a variety of sizes. FIG. 4 shows the degrees of circular to ellipticity supporting wounds of a variety of sizes. The said device is durable for cleaning with antimicrobial solvents and solutions and sterilization methods.

I claim:

1. A dome bandage is a reusable device to enclose skin wound cut(s), abrasion(s), rash(s), ulcer(s), biister(s), suture(s), burn(s), chafed skin, puncture(s), insect bite(s), impact wound(s), wart(s) or the like comprising of translucent water impermeable optically transparent clear ultraviolet resistant synthetic hard shell domed shaped mold with two or more slits through the flange.

2. A dome bandage in accordance with claim 1 wherein the said dome size and shape in degrees from circular to ellipticity.

3. A dome bandage in accordance with claim 1 wherein the said dome size and shape in degrees from squareness to oblongness.

4. A dome bandage in accordance with claim 1 wherein the said dome shields, guards and blocks external ultraviolet from penetrating the exterior wall.

5. A dome bandage in accordance with claim 1 wherein the corresponding slit(s) accepts any required length of porous ribbon tape to wrap, tighten, loosen and adjustably fastenable around extremities.

6. A dome bandage in accordance with claim 2, claim 3 and claim 5 wherein the flange appends on an uninjured epidermis surface without physical contact with skin wound.

7. A dome bandage in accordance with claim 2, claim 3 and claim 5 wherein the said dome interior wall encloses the injured epidermis without physical contact with skin wound.

8. A dome bandage in accordance with claim 1 and claim 5 wherein the flange will not create a hermetic seal.

9. A dome bandage in accordance with claim 1 and claim 5 wherein the said enclosed skin wound is ocularly and optically viewable.

10. A dome bandage in accordance with claim 1 and claim 7 wherein the said dome enclosed skin wound is shielded, and guarded from making physical contact with external objects.

11. A dome bandage in accordance with claim 1 wherein the said dome is durable to withstand aseptic cleaning solvents, acids and base solutions to be cleaned and reused.

12. A dome bandage in accordance with claim 1 and claim 10 wherein the said dome is shatter proof.
